(19) United States
(12) Patent Application Publication
(10) Pub. No.: US 2026/0094330 A1
(43) Pub. Date: Apr. 2, 2026

(54) METHOD FOR RECONSTRUCTING X-RAY IMAGE DATA, METHOD FOR PROVIDING A TRAINED MODEL, PROCESSING DEVICE, X-RAY APPARATUS, COMPUTER PROGRAM, AND DATA STORAGE MEDIUM

(71) Applicant: Siemens Healthineers AG, Forchheim (DE)

(72) Inventor: Annette Birkhold, Stuttgart (DE)

(21) Appl. No.: 19/344,509

(22) Filed: Sep. 29, 2025

(30) Foreign Application Priority Data
Sep. 30, 2024 (DE) 10 2024 209 490.0

(51) Int. Cl. G06T 11/00; G06T 7/00; G06V 10/12; G06V 10/25; G16H 30/20; G16H 30/40

(52) U.S. Cl. CPC G06T 12/00; G06T 7/0016; G06V 10/12; G06V 10/25; G16H 30/20; G16H 30/40; G06T 2207/10081; G06T 2207/20081; G06T 2207/30101; G06V 2201/03

(57) ABSTRACT

A computer-implemented method for reconstructing three-dimensional or four-dimensional X-ray image data includes receiving a first group of two-dimensional X-ray images that each depict at least part of a relevant segment of a vascular system of a patient. A three-dimensional relevant region of the patient that includes the relevant segment (4) of the vascular system of the patient is automatically determined by processing the X-ray images from the first group by an analysis algorithm. The three-dimensional or four-dimensional X-ray image data is reconstructed based on the first group of the two-dimensional X-ray images and/or a received second group of X-ray images of the patient such that all voxels of the X-ray image data are located inside the determined relevant region.

This application claims the benefit of German Patent Application No. DE 10 2024 209 490.0, filed on Sep. 30, 2024, which is hereby incorporated by reference in its entirety.

### BACKGROUND

The present embodiments relate to a computer-implemented method for reconstructing three-dimensional or four-dimensional X-ray image data, a computer-implemented method for providing a model trained by machine learning, a processing device, an X-ray apparatus, a computer program, and a data storage medium.

In three-dimensional or four-dimensional X-ray imaging (e.g., in subtraction angiography of a vascular system), the achievable level of detail or the achievable resolution of the X-ray image data is limited in many usage cases not by the imaging of the underlying two-dimensional X-ray images itself but by the voxel resolution of the reconstructed X-ray image data. The voxel resolution is fixed in standard reconstruction approaches.

Since a high level of detail is to be provided for robust identification of relevant features in the X-ray image data (e.g., for diagnosing neurovascular conditions, such as an arteriovenous malformation or an aneurysm), a multi-stage procedure, in which, after capturing all the relevant X-ray images, a provisional reconstruction is made of the entire area that may be reconstructed based on the X-ray images has been standard practice until now. Based on this provisional reconstruction, medical professionals then select a relevant region (e.g., a region that depicts the relevant area of the vascular system), after which the reconstruction is repeated for this relevant area in order to achieve an optimum resolution here.

This procedure leads to an additional workload for medical professionals and may constitute a potential source of errors, because in some cases, areas relevant to subsequent automatic image-data processing may lie outside the manually segmented area. For example, in four-dimensional digital subtraction angiography, the entire contrast-agent enhanced portion of the vascular structure is to lie in the relevant region selected for the reconstruction for every time step, because otherwise, an X-ray attenuation image through vascular segments containing contrast agent and lying outside the reconstructed area cannot be assigned to the correct vessel and hence falsifies the image values in the reconstructed volume.

In addition, reconstructing three-dimensional or even four-dimensional X-ray image data is very computationally intensive. The requirement to perform two complete reconstructions, with a user needing to assess the result of the first reconstruction between these two reconstructions, therefore demands the provision of high computing power in order to allow medical professionals to work smoothly. This results in additional costs for an imaging apparatus being used or for another computing apparatus (e.g., a workstation computer, a server, or a Cloud solution) that is used for the reconstruction.

As an alternative to the multi-stage procedure described, higher levels of detail may be achieved in principle by reconstructing higher-resolution X-ray image data directly. In order to adjust the resolution of the X-ray image data, however, the reconstruction algorithm may be modified significantly (e.g., by adjusting the size of filter kernels or other matrices). An increase in resolution leads to a disproportionate increase in the computational effort, resulting in even higher hardware costs or an even longer processing time than the above-described procedure.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an improved approach for reconstructing X-ray image data relating to a vascular system is provided.

In one embodiment, a computer-implemented method for reconstructing three-dimensional or four-dimensional X-ray image data includes the following steps: receiving a first group of two-dimensional X-ray images that each depict at least part of a relevant segment of a vascular system of a patient; automatically determining a three-dimensional relevant region of the patient that includes the relevant segment of the vascular system of the patient by processing the X-ray images from the first group by an analysis algorithm; reconstructing the three-dimensional or four-dimensional X-ray image data based on the first group of the two-dimensional X-ray images and/or a received second group of X-ray images of the patient such that all voxels of the X-ray image data are located inside the determined relevant region.

The automatic determination of the relevant region of the patient provides that it is not necessary for medical professionals to select manually the relevant region in order to achieve an optimum resolution inside the relevant region. As a result, the burden on medical professionals may be relieved and potential operating errors may be avoided. As will be explained later in greater detail, given a suitable implementation of the analysis algorithm, this may require far less computational effort than the additional reconstruction required to locate the relevant region manually at initially lower spatial resolution. Thus, the procedure according to the present embodiments may not only relieve the burden on medical professionals but may also significantly reduce the computational effort required to provide the high resolution reconstruction compared with the above-described procedure, so that a result of the same quality may be provided more quickly with the same hardware costs or for the same available computing time with low hardware costs.

The voxel resolution of the three-dimensional or four-dimensional X-ray image data may be, at least in one or two of the spatial dimensions or in all the spatial dimensions, independent of the dimensions of the determined relevant region. As was already explained in the introductory part, standard reconstruction algorithms may specify a fixed resolution for the reconstructed X-ray image data or, for example, for individual planes of the reconstructed X-ray image data. It is possible that as part of the automatic determination of the relevant region, different dimensions are determined for all the spatial dimensions (e.g., for the height, the width, and the depth) of the relevant region. It is also possible, however, that it is stipulated that the determined relevant region has the same dimension for two or three of the spatial dimensions (e.g., has the shape of a cuboid with a square base or a cube shape).

The number of X-ray images in the first group of X-ray images may be lower at least by a factor of two or at least by a factor of four than the number of X-ray images used to reconstruct the three-dimensional or four-dimensional X-ray image data. Alternatively or additionally, the number of X-ray images in the first group of X-ray images may be at most six or exactly three or exactly two.

For example, the reconstruction of the three-dimensional or four-dimensional X-ray image data may be performed based on the first group of X-ray images and the second group of X-ray images, where the second group is at least as large (e.g., at least three times as large) as the first group. When reconstructing four-dimensional X-ray image data, the number of X-ray images in the first group of X-ray images may be smaller by a specified factor than the number of X-ray images used to reconstruct the four-dimensional X-ray image data, where the specified factor is at least twice as large or at least four times as large as the number of resolved time steps in the four-dimensional X-ray image data.

During development of the present embodiments, it was found that by using a suitable analysis algorithm to identify the relevant region of the patient, only a few X-ray images are required in typical usage cases, and, for example, far fewer X-ray images are required than are needed for a high-quality reconstruction. Hence, the analysis algorithm is to process only a small quantity of input data, resulting in a required memory capacity and typically also a computational effort for performing the analysis algorithm that are far lower than for performing an additional reconstruction. After this, it would be possible to select the relevant region (e.g., manually or by automatic segmentation in three-dimensional space). In one embodiment, in the method, the X-ray images in the first group are thus processed directly or after purely two-dimensional preprocessing by the analysis algorithm. In one embodiment, no reconstructions of three-dimensional or four-dimensional image data takes place within the analysis algorithm.

A model trained by machine learning may be used as the analysis algorithm or as a sub-algorithm of the analysis algorithm. A “model trained by machine learning” may also be referred to as a “trained function.”

In general, a model trained by machine learning mimics cognitive functions that humans associate with the minds of other humans. For example, by training based on training data, the trained model is capable of adapting to new circumstances and recognizing and extrapolating patterns. Hence, such a trained model, after suitable training, may already recognize quasi-intuitively by analyzing a few projection images (e.g., just a front view and a side view of the patient or of the imaged area) the boundaries or dimensions that the relevant region of the patient is to have in order to depict the relevant segment of the vascular system in full.

In general, parameters of the model may be adapted by training. For example, supervised learning, semi-supervised learning, unsupervised learning, reinforcement learning, and/or active learning may be used. In addition, representation learning (also known as feature learning) may be employed. For example, the parameters of the trained model may be adapted iteratively by a plurality of training steps. For example, a specified cost function may be minimized in the training. For example, when training a neural network, error feedback may be used for the training.

In the method according to the present embodiments, a model that is trained by supervised learning may be used. The training datasets used include, for example, the X-ray images in the first group as the input data for the model to be trained, and define the relevant region of the patient as the desired output data for the processing of the input data of the training dataset. The desired output data specified in each training dataset, and hence the output data for which the model is trained to output, may be, for example, the coordinates of the center point of the relevant region and its dimensions and/or coordinates of a bounding surface of the relevant region.

Additional requirements placed on the determined relevant region (e.g., on the output data; a requirement that two or three of the spatial dimensions of the relevant region are the same) may be taken into account as an additional boundary condition in the training (e.g., as an additional term of the cost function). It is often simpler, however, to ascertain, using the trained model, first a provisional relevant region, the dimensions of which may then be adjusted in order to satisfy a relevant boundary condition. For example, if the width and depth of the relevant region are meant to be the same, the higher of these two dimensions of the provisional relevant region may be selected for both dimensions.

The output data from the particular training dataset (e.g., the boundaries or dimensions of the relevant region) may be determined as part of the preparation for the training (e.g., manually by medical professionals or by another algorithm) that, for example, may be more computationally or memory intensive than the analysis algorithm. For example, first, a reconstruction of three-dimensional or four-dimensional X-ray data may be performed based on the X-ray images in the first group and, for example, further X-ray images that were acquired jointly with these on the same patient. After this, the output data for the particular training dataset may be determined (e.g., by manual or automatic segmentation of this three-dimensional or four-dimensional X-ray image data).

A function trained by machine learning may be based, for example, on a neural network, a support vector machine (SVM), a decision tree, and/or a Bayes network, and/or on k-means clustering and/or a transformer, Q-learning, genetic algorithms, and/or association rules. For example, a neural network may be a deep neural network, a convolutional neural network (CNN), or a deep CNN. In addition, the neural network may be an adversarial network, a deep adversarial network, and/or a generative adversarial network (GAN).

The training of the model trained by machine learning may be performed outside the claimed computer-implemented method. For example, the computer-implemented method may be carried out during use of medical imaging and/or in analysis of image data from such imaging (e.g., in a medical center or in a general practitioner's surgery). The training of the model may be performed separately therefrom in terms of location and time, and/or by other people (e.g., by a manufacturer during production or development of an imaging apparatus that is used). In principle, however, it would also be possible to include the training as additional method acts in the computer-implemented method according to the present embodiments.

At least during the capture of the respective X-ray images in the second group, a collimator may be arranged between an X-ray source used to capture the X-ray image and the patient. The collimator is set for capturing the respective X-ray images in the second group according to the determined relevant region of the patient.

A collimator used in X-ray imaging may be a diaphragm that screens the X-ray radiation from the X-ray source outside a transmission area. For example, a one-dimensional collimator that restricts the width of an X-ray cone using two substantially parallel rectangular diaphragms may be used, whereby the area of the patient that is exposed to the X-ray radiation can be set. Alternatively, for example, an iris diaphragm or the like may be used as the collimator.

Since the relevant region may be determined already from the X-ray images in the first group, and areas of the X-ray images in the second group that do not affect the reconstruction of the relevant region have no relevance for the reconstructed X-ray image data, the radiation exposure of the patient during the X-ray imaging may be reduced by such a collimator setting.

For the purpose of determining the X-ray images in the second group, the collimator may be set automatically (e.g., by an actuator assigned to the collimator) according to the determined relevant region of the patient. In principle, however, it would also be possible to output the particular collimator adjustment to a user, who then sets the collimator manually according to this specification. Since in the described case, X-ray images in the second group are captured, the reconstruction of the three-dimensional or four-dimensional X-ray image data may then be performed based on the first group of X-ray images and the second group of X-ray images or based on the second group of X-ray images.

The respective X-ray images in the second group may be determined using an acquisition geometry specified for each. The collimator is set to capture the respective X-ray images in the second group additionally according to the associated acquisition geometry. While it would be possible in principle to use the same collimator setting for all the second X-ray images (e.g., by setting the width of a slit bounded by the collimator according to the maximum dimension of the determined relevant region), a separate setting for the respective X-ray images in the second group is advantageous, because stronger collimation and hence a further reduction in the radiation exposure of the patient may typically be achieved thereby for at least some of these X-ray images.

The specified acquisition geometry of the respective X-ray images in the second group may specify the location of a collimator plane in which the collimator acts as a diaphragm for the X-ray radiation from the X-ray source. The relevant region is projected onto the collimator plane in order to ascertain the collimator setting of the collimator for acquiring the respective X-ray images.

The projection may be made according to a known beam geometry of the X-ray radiation emitted by the X-ray source. The collimator may be set such that areas of the collimator plane through which the X-ray radiation potentially passes, and which lie outside the area onto which the relevant region is projected, is obscured as far as possible by a collimator component of the collimator, whereas areas onto which the relevant region is projected remain unobstructed by collimator components.

The X-ray images in the first group and/or the second group may be captured, for example, as part of digital subtraction angiography. The four-dimensional X-ray image data may depict in this case a variation over time in a local contrast agent concentration in the relevant segment of the vascular system of the patient.

The relevant region may be determined as an area that entirely encompasses the vascular system inside the head or an organ of the patient. The organ depicted may be, for example, the brain, the liver, the heart, or the lungs of the patient.

The present embodiments also relate to a computer-implemented method for providing a model trained by machine learning for use as an analysis algorithm or as a sub-algorithm of the analysis algorithm in the method according to the present embodiments for reconstructing three-dimensional or four-dimensional X-ray image data. The method includes the following steps: receiving a plurality of training datasets that each include, as input data, a plurality of X-ray images of a particular patient, each depicting at least part of a relevant segment of a vascular system of the particular patient, and as the target result, a definition of a three-dimensional relevant region, inside which the relevant segment of the vascular system of the patient is located; training a model based on the training datasets to determine the model trained by machine learning; and providing the model trained by machine learning.

The described embodiment of the training datasets allows supervised learning. To do this, starting from an initial (e.g., random) parameterization of the model to be trained, the parameterization may be iteratively adjusted in order to minimize a deviation of a three-dimensional relevant region determined based on the X-ray images of the particular training dataset from the definition of this region specified as target results in the training dataset. For example, a cost function may be or include a sum of measures of differences between the dimensions and/or coordinates of the region determined by the model and the dimensions and/or coordinates of the region defined by the associated target result. This cost function may then be minimized (e.g., using a gradient descent method) by varying the parameters of the model with error feedback. Approaches for providing suitable training datasets have already been described above.

The present embodiments also relate to a processing device that is configured to perform the computer-implemented method according to the present embodiments for reconstructing three-dimensional or four-dimensional X-ray image data and/or the computer-implemented method according to the present embodiments for providing a model trained by machine learning.

The processing device may be, for example, suitably programmed data processing apparatuses, or at least some of the aforementioned functionality may alternatively have a hard-wired implementation. The processing device may be integrated in a medical imaging apparatus (e.g., an X-ray apparatus) or be separate therefrom. For example, the processing device may be implemented as a workstation computer, server, or Cloud solution.

The present embodiments also relate to an X-ray apparatus including an X-ray source and an X-ray detector for determining X-ray images of a patient and also a processing device according to the present embodiments. Integrating the processing device according to the present embodiments in the X-ray apparatus may further simplify and speed up the workflow for X-ray imaging.

The present embodiments also relate to a computer program containing instructions that are configured to perform, when executed on a data processing apparatus, the computer-implemented method according to the present embodiments for reconstructing three-dimensional or four-dimensional X-ray image data and/or the computer-implemented method according to the present embodiments for providing a model trained by machine learning.

The present embodiments also relate to a data storage medium including the computer program according to the present embodiments.

Independent of the grammatical term usage, individuals with male, female, or other gender identities are included within the term.

### BRIEF DESCRIPTION OF THE DRAWINGS

Further advantages and details of the present embodiments are presented in the following example embodiments and in the associated drawings, in which:

FIG. 1 schematically shows an example embodiment of an X-ray apparatus that includes an example embodiment of a processing device;

FIG. 2 schematically shows an example embodiment of a method for reconstructing three-dimensional or four-dimensional X-ray image data;

FIG. 3 schematically shows examples of X-ray images in a first group in the method shown in FIG. 2, and a provisional relevant region determined therefrom;

FIG. 4 schematically shows a flow diagram of an example embodiment of a method for providing a model trained by machine learning; and

FIG. 5 schematically shows the structure of an example of a model trained by machine learning that may be used in the methods according to FIGS. 2 and 4.

### DETAILED DESCRIPTION

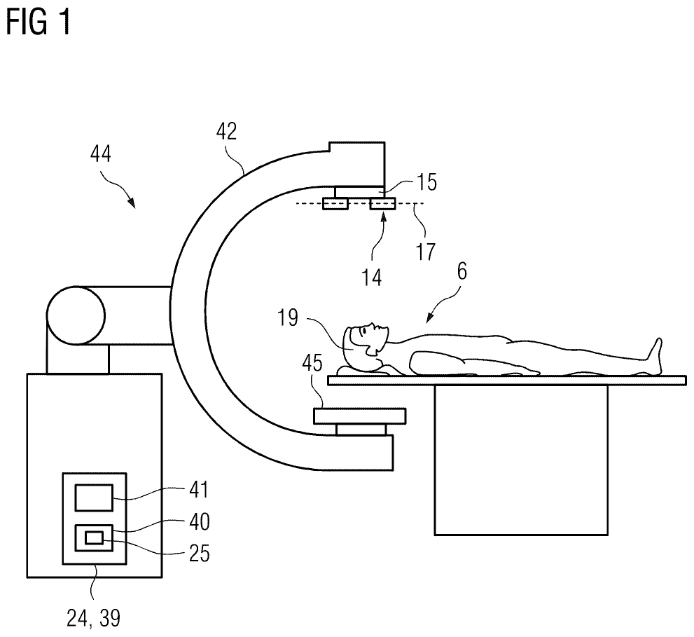

FIG. 1 shows an X-ray apparatus 44 for capturing X-ray images of a patient 6, with an area of a head 19 of a patient 6 being captured in the example shown. X-ray images may be acquired from different perspectives in order to capture a reconstruction of three-dimensional image data (e.g., for digital subtraction angiography, such as for four-dimensional digital subtraction angiography).

As was already explained in the general part of the description, a reconstruction of the three-dimensional or four-dimensional X-ray image data 1 may be performed, for example, with a fixed voxel resolution, which provides that the achievable spatial resolution falls as the size of the depicted region increases. In order to provide that the entire relevant segment 4 of the vascular system (e.g., the entire area suffused with contrast agent during the digital subtraction angiography) may be depicted, the imaging may be performed, such that in a standard reconstruction of the X-ray image data 1, a region of the patient that is larger than a relevant region 7 of the patient 6, which includes the relevant segment 4 of the vascular system 5 of the patient 6, may be depicted. This results in a lower spatial resolution.

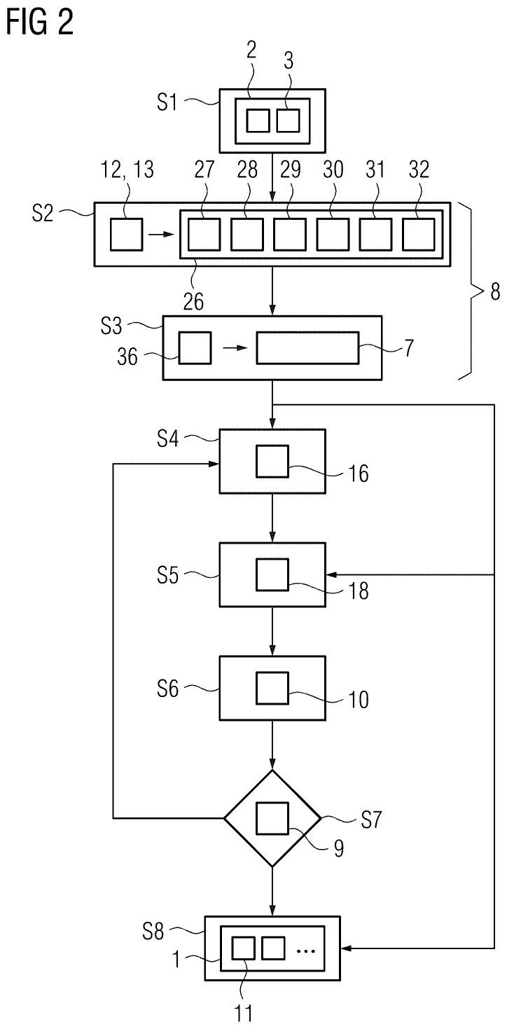

In order to achieve an optimum spatial resolution, the X-ray apparatus 44 therefore includes a processing device 39 that is configured to perform a computer-implemented method for reconstructing three-dimensional or four-dimensional X-ray image data, which is explained in greater detail below with reference to FIG. 2 and in which a plurality of (e.g., two) X-ray images 3 are processed by an analysis algorithm 8 in order to determine automatically the three-dimensional relevant region 7 of the patient 6, which includes the relevant segment 4 of the vascular system 5 of the patient 6. The reconstruction of the three-dimensional or four-dimensional X-ray image data 1 is then performed based on these two-dimensional X-ray images 3 or, for example, alternatively or additionally based on a second group 9 of X-ray images 10 of the patient 6 such that all the voxels 11 of the X-ray image data 1 are located inside the determined relevant region 7.

The advantages of such a procedure are explained by way of example for imaging the vascular system in the head 19 of the patient 6. In a standard imaging geometry used for this purpose, a standard reconstruction may result in a dimension of the reconstructed area of 248 mm in each spatial dimension, for example. Given a resolution of 1024 voxels in each direction, a spatial resolution of 0.24 mm is achieved. If now the dimension of the relevant region in the width direction and in the depth direction of the patient equals 16.4 cm and 12 cm, respectively, for example, then even when square voxels are meant to be used in this plane, the reconstructed region may be restricted to dimensions of 16.4 cm. A resolution of 0.16 mm results, and thus, a 33% higher resolution may be achieved.

In the example shown in FIG. 1, the processing device 39 is implemented by a programmable data processing apparatus 24, the processor 41 of which executes the instructions of a computer program 25 implementing the method. The computer program 25 is stored on a data storage medium 40 (e.g., a non-transitory computer-readable storage medium).

An example embodiment of such a method for reconstructing X-ray image data 1 is explained below with additional reference to the flow diagram shown in FIG. 2.

In act S1, two-dimensional X-ray images 3 are first captured in a first group 2. Alternatively, for example, these X-ray images may be received from another device or read from a memory.

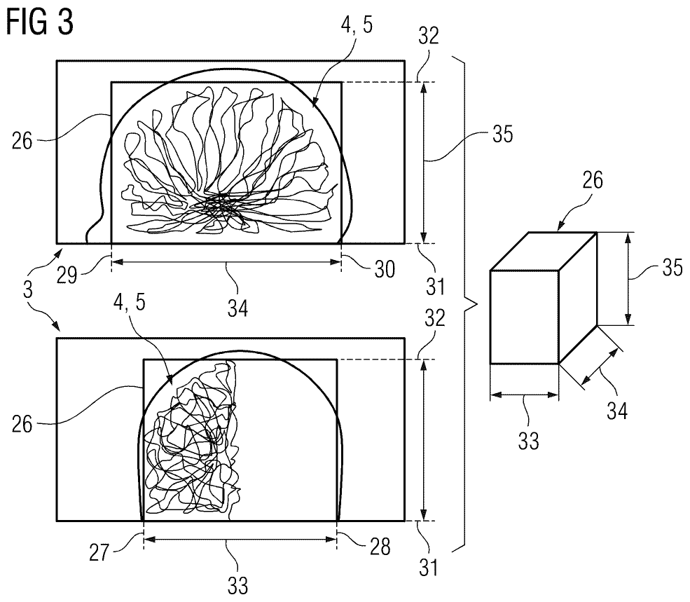

As shown schematically in FIG. 3, just two X-ray images 3 are taken into account in the example, which have acquisition geometries that are substantially orthogonal to each other. The upper of the X-ray images 3 shown in FIG. 3 was obtained by a lateral acquisition with a C-arm 42 rotated out of the picture plane through 90° compared with the position shown in FIG. 1. The lower of the two X-ray images 3 shown in FIG. 3 is captured in an anterior-posterior perspective (e.g., with the imaging geometry presented in FIG. 1).

FIG. 3 shows X-ray images 3 for better identification of the vascular system 5. In the X-ray images 3, the vascular system 5 in the right half of the head or brain is suffused with contrast agent and hence clearly identifiable. If the described method is used for reconstruction in four-dimensional digital subtraction angiography, it may be advantageous in actual use if X-ray acquisitions that are captured at the start of the X-ray sequence are used as X-ray images 3 in the first group, whereby typically little or no contrast agent is yet present in the vascular system 5. This may be provided because then the relevant area 7 determined based on these X-ray images 3 may be used in the collimation of the subsequent X-ray acquisitions 10 of the sequence, as will be explained later.

The acts S2 and S3 implement the analysis algorithm 8. In the example, the X-ray images 3 in the first group 2 are processed in act S2 initially by a sub-algorithm 12 of the analysis algorithm 8 in order to determine a provisional relevant region 26. The provisional relevant region is defined in the example by the positions 27-32 of bounding surfaces of the provisional relevant region 26 in the different spatial directions, which are presented in FIG. 3

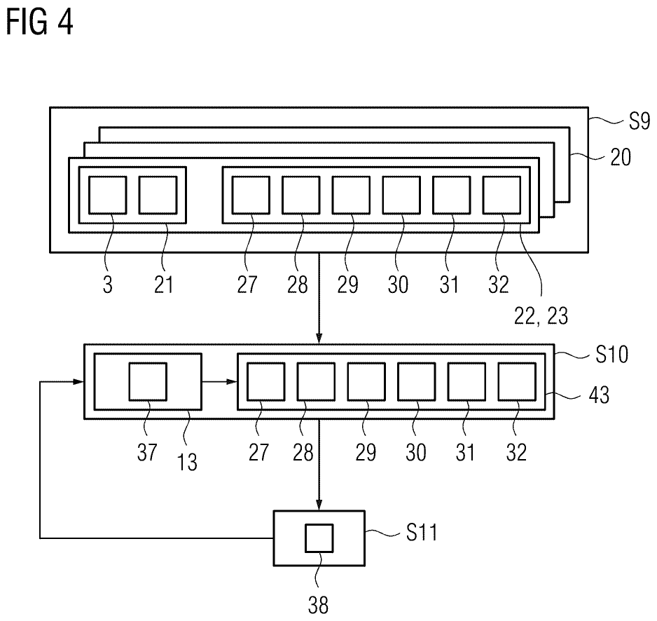

In principle, it would be possible to determine the, or the provisional, relevant region 7, 26 based on segmentation of the vascular system 4 in the X-ray images in the first group 2. Automatic segmentation of the vascular system 5 in the X-ray images 3 in the first group 2 may not be possible robustly using classical segmentation approaches, however, for example, when the vascular system in the X-ray images 3 in the first group 2 is still largely free of contrast agent. Therefore, in the example shown, a model 13 trained by machine learning is used as the sub-algorithm 12 in order to achieve robust segmentation even when a vascular system does not contain contrast agent. A possible way of training such a model 13 will be explained later with reference to FIG. 4.

As shown in FIG. 3, in the example, different dimensions 33-35 of the provisional relevant region 26 result for the different spatial directions. Since, in the example, a square shape of each voxel of the reconstructed X-ray image data is meant to be achieved in the plane spanned by the dimensions 33, 34, a boundary condition 36 is applied in act S3, according to which the smaller of the dimensions 33, 34 is set to the value of the larger of these dimensions 33, 34 in order to define the relevant region 26 used for the reconstruction. Depending on the specific implementation of the reconstruction and the visualization of the reconstructed data, this act may potentially be omitted in order to achieve potentially a higher level of detail of the reconstructed X-ray image data.

In acts S4-S7, further X-ray images are then captured, which are associated with a second group 9. The further X-ray images 10 are used, for example, both to provide additional imaging geometries for a high-quality reconstruction and to capture image data at different times during the suffusion of contrast agent in the vascular system 5 and hence perform four-dimensional digital subtraction angiography.

In act S4, the desired acquisition geometry 16 is first set for the particular X-ray acquisition 10 (e.g., by tilting and/or rotating the C-arm 42).

Then, in act S5, or alternatively even while setting the acquisition geometry 16, a suitable collimator setting 18 of a collimator 14 arranged between the X-ray source 15 and the patient 16 may be determined, and the collimator 14 may be set accordingly. For this purpose, the relevant region 7 determined in act S3 is projected onto the collimator plane 17, shown in FIG. 1, based on the known beam geometry of the radiation source 15 and the imaging geometry 16. The rectangular diaphragms, which in the example form the collimator 14, are then set such that that surface on which the relevant region 7 is reproduced under this projection is unobstructed, whereas areas outside this surface are obscured, as far as possible, by the diaphragms in order to minimize the radiation dose to which the patient 6 is exposed during the imaging.

In act S6, the X-ray source 15 is then activated for imaging in order to acquire, using the X-ray detector 45, an associated X-ray image 10 in the second group 9. Then, in act S7, it is checked whether all the X-ray images 10 in the second group 9 have already been captured. If this is not the case, the method is repeated from act S4 for the next X-ray image 10 to be acquired in the second group 9.

After capturing all the X-ray images 10 in the second group 9, the X-ray image data 1 is reconstructed in act S8 such that all the voxels 11 of the X-ray image data 1 are located inside the determined relevant region 7. A number of approaches are known for reconstructing the three-dimensional image data of a specific volume based on projection images that depict this volume at least in part. These approaches may be used in act S8. For example, filtered backprojection, an iterative reconstruction method, or reconstruction using a further model trained by machine learning may be performed.

The described procedure achieves an at least approximately optimum resolution of the reconstructed X-ray image data 1 without the need for complex user interactions and with less computational effort than would be required in the multiple reconstruction explained at the beginning of the general part of the description. In addition, the described automatic collimator setting may reduce the X-ray dose to which the patient 6 is exposed for the imaging compared with standard imaging approaches.

FIG. 4 shows a flow diagram of a method for providing a model 13 trained by machine learning, which may be used in act S2 of the method shown in FIG. 2 for determining the provisional relevant region 26. In principle, the method may also be performed by the processing device 39 shown in FIG. 1. For training the model 13, however, a separate processing device (not shown) (e.g., a server or a Cloud solution) may be used. One reason for doing so is that this training is typically performed by people other than those using the algorithm shown in FIG. 2. For example, the training may be performed by a manufacturer of the X-ray apparatus 44 shown in FIG. 1.

In act S9, a plurality of training datasets 20 are received. Each training dataset of the plurality of training datasets 20 includes, as input data 21, a plurality of X-ray images 3 of a particular patient 6. Each X-ray image of the plurality of X-ray images 3 depicts at least part of a relevant segment 4 of a vascular system 5 of the particular patient 6. Each training dataset of the plurality of training datasets 20 also includes, as the target result 23, a definition 22 of a three-dimensional relevant region, inside which the relevant segment 4 of the vascular system 5 of the patient 6 is located. In the example, the definition 22 is made by specifying the positions 27-32 of bounding surfaces of the relevant region. These bounding surfaces may be determined, for example, by reconstructing, based on a dataset of X-ray images, from which the X-ray images 3 originate, three-dimensional X-ray image data, in which segmentation of the relevant region is then performed automatically or manually by medical professionals.

In act S10, the model 13 is then applied using its initial or current parameterization 37 to the X-ray images 3 of the input data 21 in order to determine an actual result 43 for the relevant region. In the example, the actual result includes actual values of each of the positions 27-32 of bounding surfaces of the relevant region.

In act S11, a cost function 38 may thus be evaluated. The cost function 38 is a sum of measures of the deviation of each position 27-32 in the actual result 43 from the corresponding position 27-31 in the target result 23. The parameterization 37 of the trained function 13 may then be adjusted by error feedback known per se. For example, a gradient descent method may be used for minimizing the cost function 38.

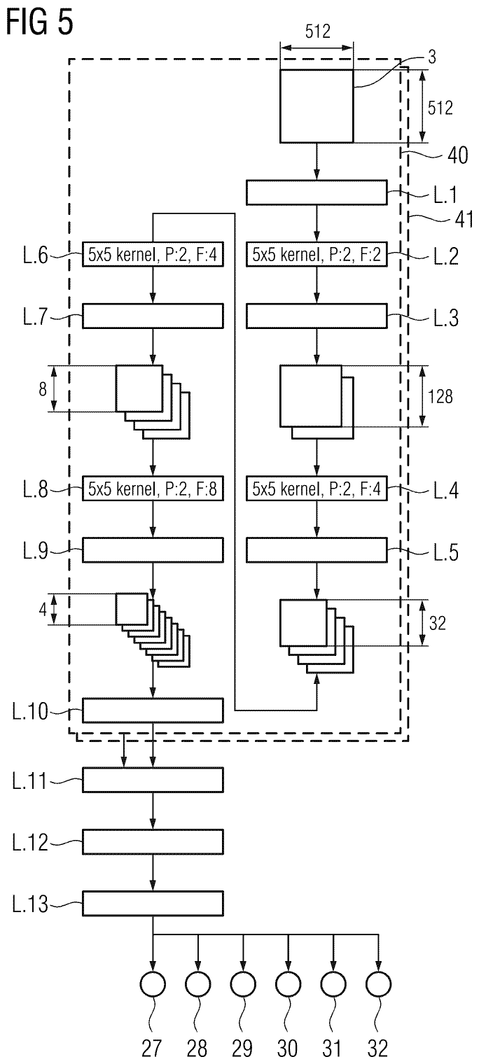

The trained model 13 used may be, for example, the convolutional neural network shown in FIG. 5. The layers L.1 are L.10 are duplicated, once in the subnetwork 40 shown and again in the subnetwork 41, where the layer L.11 processes as input data the output data from the respective layers L.10 in the subnetwork 40 and the subnetwork 41. The subnetworks 40, 41, or more precisely their respective layers L.1, each process one of the X-ray images 3 in the first group 2. Thus, in the example shown, first, separate preprocessing of the X-ray images 3 takes place in the layers L.1-L.10, after which the layers L.11-L.13 perform joint processing of the resulting interim results.

The neural network in the example consists of convolutional layers, pooling layers, and fully connected layers. In the input layer L.1, there is a node for each pixel of the associated input data, with each pixel having a channel (e.g., the associated intensity value). After the input layer come four convolutional layers L.2, L.4, L.6, L.8, with each of the four convolutional layers followed by a pooling layer L.3, L.5, L.7, L.9. For each of the convolutional layers, a 5×5 kernel is used (indicated by “5×5 kernel”) with a padding of 2 (indicated by “P: 2”) and an increasing number of filters/convolution kernels (indicated by “F: 2”, “F: 4” or “F: 8”). In addition, there are four pooling layers L.3, L.5, L.7, L.9, where the first three layers L.3, L.5, L.7 perform an averaging across fields of size 4×4, and the last pooling layer L.9 performs a maximum selection across fields of size 2×2. FIG. 5 shows an additional layer L.10 that flattens the input images (e.g., combines 8 images of size 4×4 into a vector of 128 entries). This layer, however, is not relevant to the actual calculation.

The last layers of the network are three fully connected layers L.11, L.12, L.13, where the first fully connected layer has 256 input nodes and 64 output nodes, the second fully connected layer L.12 has 64 input nodes and 24 output nodes, and the third fully connected layer L.13 has 24 input nodes and 6 output nodes, with each of the output nodes providing one of the positions 27-32 that define the provisional relevant region 26 in the example.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for reconstructing three-dimensional or four-dimensional X-ray image data, the method being computer-implemented and comprising:
receiving a first group of two-dimensional X-ray images, each of the two-dimensional X-ray images of the first group depicting at least part of a relevant segment of a vascular system of a patient;
automatically determining a three-dimensional relevant region of the patient that comprises the relevant segment of the vascular system of the patient, the automatically determining comprising processing the two-dimensional X-ray images from the first group by an analysis algorithm; and
reconstructing the three-dimensional or four-dimensional X-ray image data based on the first group of the two-dimensional X-ray images, a received second group of X-ray images of the patient, or a combination thereof, such that all voxels of the three-dimensional or four-dimensional X-ray image data are located inside the determined three-dimensional relevant region.

2. The method of claim 1, wherein:
a number of X-ray images in the first group of two-dimensional X-ray images is lower at least by a factor of two or at least by a factor of four than a number of X-ray images used to reconstruct the three-dimensional or four-dimensional X-ray image data;
the number of X-ray images in the first group of two-dimensional X-ray images is at most six, exactly three, or exactly two; or
a combination thereof.

3. The method of claim 1, wherein a model trained by machine learning is used as the analysis algorithm or as a sub-algorithm of the analysis algorithm.

4. The method of claim 1, wherein at least during capture of the respective X-ray images in the second group, a collimator is arranged between an X-ray source used to capture the X-ray image and the patient, and
wherein the collimator is configured to capture the respective X-ray images in the second group according to the determined relevant region of the patient.

5. The method of claim 4, wherein the respective X-ray images in the second group are determined using an acquisition geometry specified for each, and
wherein the collimator is configured to capture the respective X-ray images in the second group additionally according to the associated acquisition geometry.

6. The method of claim 5, wherein the specified acquisition geometry of the respective X-ray images in the second group specifies a location of a collimator plane in which the collimator acts as a diaphragm for X-ray radiation from the X-ray source,
wherein the relevant region is projected onto the collimator plane, such that a collimator setting of the collimator for acquiring the respective X-ray images is ascertained.

7. The method of claim 1, wherein the two-dimensional X-ray images in the first group, the X-ray images in the second group, or the two-dimensional X-ray images in the first group and the X-ray images in the second group are captured as part of digital subtraction angiography.

8. The method of claim 7, wherein the four-dimensional X-ray image data depicts a variation over time in a local contrast agent concentration in the relevant segment of the vascular system of the patient.

9. The method of claim 1, wherein the relevant region is determined as an area that entirely encompasses the vascular system inside the head or an organ of the patient.

10. A method for providing a model trained by machine learning for use as an analysis algorithm or as a sub-algorithm of the analysis algorithm, the method being computer-implemented and comprising:
receiving a plurality of training datasets, each training dataset of the plurality of training datasets comprising, as input data, a plurality of X-ray images of a particular patient, each X-ray image of the plurality of X-ray images depicting at least part of a relevant segment of a vascular system of the particular patient, and as a target result, a definition of a three-dimensional relevant region, inside which the relevant segment of the vascular system of the patient is located;
training a model based on the plurality of training datasets, such that the model trained by machine learning is determined; and
providing the model trained by machine learning.

11. A processing device comprising:
a processor configured to reconstruct three-dimensional or four-dimensional X-ray image data, the processor being configured to reconstruct the three-dimensional or four-dimensional X-ray image data comprising the processor being configured to:
receive a first group of two-dimensional X-ray images, each of the two-dimensional X-ray images of the first group depicting at least part of a relevant segment of a vascular system of a patient;
automatically determine a three-dimensional relevant region of the patient that comprises the relevant segment of the vascular system of the patient, the automatic determination comprising processing of the two-dimensional X-ray images from the first group by an analysis algorithm;
reconstruct the three-dimensional or four-dimensional X-ray image data based on the first group of the two-dimensional X-ray images, a received second group of X-ray images of the patient, or a combination thereof, such that all voxels of the three-dimensional or four-dimensional X-ray image data are located inside the determined three-dimensional relevant region.

12. An X-ray apparatus comprising:
an X-ray source;
an X-ray detector configured to determine X-ray images of a patient; and
a processor configured to reconstruct three-dimensional or four-dimensional X-ray image data, the processor being configured to reconstruct the three-dimensional or four-dimensional X-ray image data comprising the processor being configured to:
receive a first group of two-dimensional X-ray images, each of the two-dimensional X-ray images of the first group depicting at least part of a relevant segment of a vascular system of a patient;
automatically determine a three-dimensional relevant region of the patient that comprises the relevant segment of the vascular system of the patient, the automatic determination comprising processing of the two-dimensional X-ray images from the first group by an analysis algorithm;
reconstruct the three-dimensional or four-dimensional X-ray image data based on the first group of the two-dimensional X-ray images, a received second group of X-ray images of the patient, or a combination thereof, such that all voxels of the three-dimensional or four-dimensional X-ray image data are located inside the determined three-dimensional relevant region.

13. In a non-transitory computer-readable storage medium that stores instructions executable by one or more processors to reconstruct three-dimensional or four-dimensional X-ray image data, the instructions comprising:
receiving a first group of two-dimensional X-ray images, each of the two-dimensional X-ray images of the first group depicting at least part of a relevant segment of a vascular system of a patient;
automatically determining a three-dimensional relevant region of the patient that comprises the relevant segment of the vascular system of the patient, the automatically determining comprising processing the two-dimensional X-ray images from the first group by an analysis algorithm; and
reconstructing the three-dimensional or four-dimensional X-ray image data based on the first group of the two-dimensional X-ray images, a received second group of X-ray images of the patient, or a combination thereof, such that all voxels of the three-dimensional or four-dimensional X-ray image data are located inside the determined three-dimensional relevant region.

14. The non-transitory computer-readable storage medium of claim 13, wherein:
a number of X-ray images in the first group of two-dimensional X-ray images is lower at least by a factor of two or at least by a factor of four than a number of X-ray images used to reconstruct the three-dimensional or four-dimensional X-ray image data;
the number of X-ray images in the first group of two-dimensional X-ray images is at most six, exactly three, or exactly two; or
a combination thereof.

15. The non-transitory computer-readable storage medium of claim 13, wherein a model trained by machine learning is used as the analysis algorithm or as a sub-algorithm of the analysis algorithm.

16. The non-transitory computer-readable storage medium of claim 13, wherein at least during capture of the respective X-ray images in the second group, a collimator is arranged between an X-ray source used to capture the X-ray image and the patient, and
wherein the collimator is configured to capture the respective X-ray images in the second group according to the determined relevant region of the patient.

17. The non-transitory computer-readable storage medium of claim 16, wherein the respective X-ray images in the second group are determined using an acquisition geometry specified for each, and
wherein the collimator is configured to capture the respective X-ray images in the second group additionally according to the associated acquisition geometry.

18. The non-transitory computer-readable storage medium of claim 17, wherein the specified acquisition geometry of the respective X-ray images in the second group specifies a location of a collimator plane in which the collimator acts as a diaphragm for X-ray radiation from the X-ray source, and
wherein the relevant region is projected onto the collimator plane, such that a collimator setting of the collimator for acquiring the respective X-ray images is ascertained.

19. The non-transitory computer-readable storage medium of claim 13, wherein the two-dimensional X-ray images in the first group, the X-ray images in the second group, or the two-dimensional X-ray images in the first group and the X-ray images in the second group are captured as part of digital subtraction angiography.
